SEQUENCE LISTING (11 sequences)

SEQ ID NO: 1 - protein, 12 aa, synthetic construct
CDKTHPAPELLG

SEQ ID NO: 2 - protein, 11 aa, synthetic construct
DKTHPAPELLG

SEQ ID NO: 3 - protein, 10 aa, synthetic construct
KTHPAPELLG

SEQ ID NO: 4 - protein, 9 aa, synthetic construct
THPAPELLG

SEQ ID NO: 5 - protein, 8 aa, synthetic construct
HPAPELLG

SEQ ID NO: 6 - protein, 7 aa, synthetic construct
PAPELLG

SEQ ID NO: 7 - protein, 6 aa, synthetic construct
APELLG

SEQ ID NO: 8 - protein, 5 aa, synthetic construct
PELLG

SEQ ID NO: 9 - protein, 4 aa, synthetic construct
ELLG

SEQ ID NO: 10 - protein, 11 aa, synthetic construct
PAPELLGGPSV

SEQ ID NO: 11 - protein, 10 aa, synthetic construct
CSKPTSPPPE

(19) United States
(12) Patent Application Publication
(10) Pub. No.: US 2026/0062496 A1
(43) Pub. Date: Mar. 5, 2026

(54) METHODS OF TREATMENT COMPRISING IGG ANTIBODIES AND AN IDES PROTEASE

(71) Applicant: ROBERT JORDAN, Thornton, PA (US)

(72) Inventor: ROBERT JORDAN, Thornton, PA (US)

(21) Appl. No.: 19/106,327

(22) PCT Filed: Sep. 11, 2023
(86) PCT No.: PCT/US2023/032410

§ 371 Date: Feb. 25, 2025

Related U.S. Application Data
(60) Provisional application No. 63/404,998, filed on Sep. 9, 2022.

(51) Int. Cl. C07K 16/28; A61K 38/00; A61K 39/00; A61P 35/00; C12N 9/64; C12P 21/02

(52) U.S. Cl. CPC C07K 16/2887; A61P 35/00; C12N 9/641; C12P 21/02; A61K 38/00; A61K 2039/505; C07K 2317/732; C12Y 304/22

(57) ABSTRACT

The present invention relates to methods of treating diseases in a subject in need thereof, including proliferative diseases such as cancer, especially solid tumors, as well as other diseases involving invasive cells or entities such as viruses, bacteria, fungi, and parasites, using IgG antibodies, including anti-tumor IgG antibodies, and a protease from Streptococcus pyogenes.

### CROSS-REFERENCE TO RELATED APPLICATION

This application is a U.S. National Stage Entry of International Patent Application No. PCT/US2023/032410 filed Sep. 11, 2023, which claims the benefit of priority to U.S. Provisional Patent Application No. 63/404,998 filed on Sep. 9, 2022, the entirety of which are incorporated herein by reference.

The present invention relates to methods of treating diseases, such as cancer, especially solid tumors, by using IgG antibodies, including anti-tumor IgG antibodies, that are modified by the protease IdsS.

### SEQUENCE LISTING

The instant application contains a Sequence Listing which has been submitted herewith and is hereby incorporated by reference in its entirety. Said .xml copy, created on Dec. 11, 2023 is named JRDN0001-100PCT-00592426, and is 10,602 bytes in size.

### BACKGROUND

The development of specific anti-tumor monoclonal antibodies (“mAbs”) came with the promise of breakthroughs in cancer immunotherapy. Through the binding of a mAb to a tumor associated antigen (“TAA”), immune effector systems of the host were expected to be activated. The pathways include natural killer (“NK”) cells (invoking antibody-dependent cellular cytotoxicity, “ADCC”), complement-dependent cytotoxicity (“CDC”) and phagocytic mechanisms. Although progress with conventional mAb treatments has been achieved in some patients, cancers in other patients proved resistant to this mAb-based approach. The findings call to mind the still poorly explained route(s) of endogenous immune evasion by emergent cancers in some individuals. From a chronic perspective, humans seem to cope more successfully with foreign bacterial infections than with mutated cells of their own origin (i.e., cancer cells).

Dr. William Coley (active primarily between 1890 and 1930) made what remain difficult-to-explain clinical observations in cancer patients. Specifically, it was observed that simultaneous S. pyogenes infections (erysipelas, scarlet fever) could be associated with dramatic reversals of sarcomas-even in the absence of surgery. Complete, and rapid remissions were occasionally reported for tumors of several centimeters in diameter. Accordingly, considerable attention was given to these findings over several decades (reviewed in Ref. 1), but the mechanism of these actions was not known. More recent attempts at mechanistic hypotheses for the benefits of Dr. Coley's findings have postulated localized immunostimulatory phenomena involving cytokines, immune cell receptor pathways, metabolic derangements in the tumor microenvironment, and other complex formulations of immune cell-based mechanisms. There is still no consensus explanation. Although Coley's concept and therapeutic approach were eclipsed by the emerging radiation and chemotherapy strategies, Coley's toxins persist as a frequently cited unsolved puzzle in cancer therapy.

Human immunity exploits multiple strategies, especially circulating antibodies (“Abs”), to combat toxic infectious pathogens and cancer cells. In some cases, pathogenic entities have developed evasive tactics-including recently-defined modes of proteolytic degradation-to deactivate host antibodies. This is a potentially very effective defensive strategy on the part of bacteria to avoid host immunity (Ref. 18) because it can bring about an inactivation of host antibody functions. Human immunity appears to have developed a specific corrective strategy to counter proteolytic inactivation of IgG antibodies (“IgGs”) as expressed by bacteria, especially S. pyogenes.

Humans are exposed to S. pyogenes shortly after birth and nearly all individuals possess IgG antibodies against the bacterium. As a potential mechanism to escape host surveillance, S. pyogenes secretes a protease, IdeS (Immunoglobulin degrading enzyme of Streptococcus pyogenes). This enzyme is inordinately potent and selective in its cleavage of human IgG. IdeS employs an exosite binding site in the human IgG Fc domain to facilitate access to the hinge and the single peptide bond that it targets (Refs. 2, 3)—a factor likely involved in its anti-human specificity. Of the two parallel heavy chains constituting the IgG hinge, the first IdeS cleavage is considerably more rapid than the second, yielding a single-cleaved IgG (“scIgG”) as the dominant product under conditions of limiting protease. Strong non-covalent Fc-Fc bonds in CH3 (Ref. 4) prevent the release of the first cleaved Fc chain from the overall structure of scIgG. Accordingly, it was found that scIgGs, rather than the double-cut F(ab′)2s, accumulate in human breast tumors. In one aspect, it needs to be emphasized that such fragments cannot derive from IdeS which is a bacterial and non-human enzyme (Refs. 5, 19). In another aspect, the scIgG is only distinguishable from unmodified IgG by using specific tools, which is the reason why scIgG's existence in vivo was unnoticed for long period of time.

Unexpectedly, the cleavage of the first of the two heavy chains results in near-total inactivation of IgG killing functions in vitro (Ref. 6). However, the antigen-targeting and cell binding actions of the distant Fab ends remain intact. These in vitro results prompted a search to see if similar events occurred in vivo. In-situ proteolysis of IgGs and the specific generation of single-cleaved IgGs was recently confirmed in certain breast cancer tumors (Refs. 5, 19). The localized presence of cleaved IgGs within tumors in vivo supports the notions that: 1) specific host antibodies against tumor antigens had been generated, and 2) that proteolytic cleavage of the host antibodies had rendered the antibodies dysfunctional to a degree such that the existing tumor had evaded eradication by host immunity (Ref. 8). The field of anti-tumor immune therapy with specific mAbs against tumor associated antigens (TAAs) is intended to compensate for inadequacies of host immunity but did not anticipate local proteolysis of tumor-bound antibodies and the resulting dysfunctions. The S. pyogenes enzyme, IdeS, is unique in that it is introduced in vivo only in the context of a bacterial infection because IdeS is not native to the human system.

Another notable observation of IgG hinge cleavage is a lack of any disruption in scIgG binding to intended targets through the Fab (fragment antigen binding) domain. The large IgG molecule is comprised of multiple, separated functional domains. Moreover, protease cleavage can proceed with the IgG bound to its cellular antigen-even preferentially compared to free IgGs in solution-ensuring that antigen-bound IgGs get cleaved in place, as was shown for matrix metalloproteinase 3 (“MMP3”)-cleaved anti-CD142 mAb on MDA-MB-23 cells (Ref. 6) and with IdeS on SKOV-3 cells (Ref. 19). An important consequence is that potential antigen sites for subsequent intact IgG binding can already be occupied by disabled Abs and it is these proteolyzed IgGs that will be shown to be at the core of the present disclosure.

The IdeS protease from S. pyogenes is an archetype for bacterial enzymes with the ability to disable host IgGs. However, IgG cleavage per se is not unique to IdeS. Extracellular proteases—although far less potent than IdeS and displaying cleavage site specificities that differ from it—are abundant during development, tissue remodeling, wound healing and in tumor growth (some of these functions are overlapping as in the case of matrix metalloproteinases). Since IgG is most likely not the primary protein substrate of such endogenous proteases, IgG cleavage could be considered a side effect or even collateral damage.

A Swedish group has characterized the enzymatic characteristics of IdeS as well as its in vivo potency in a unique clinical setting (Refs. 2, 3). Specifically, the Swedish group demonstrated that a high dose intravenous administration of purified, recombinant IdeS could deplete the entire circulating repertoire of human IgGs (˜50 grams in an adult human) (Ref. 22). The therapeutic goal of that study was to remove autoantibody populations that contribute to transplant rejection by converting them to dysfunctional and rapidly-cleared F(ab′)2 fragments (Ref. 7). A related clinical application of IdeS is also the complete removal of circulating IgGs but with the intent of relieving competition by endogenous IgGs on the FcRn (“salvage”) receptor thereby facilitating mAb therapies (Ref. 23). Those goals are opposite to the therapeutic strategy that will be outlined here. Importantly, however, Imlifidase (recombinant IdeS) is already well advanced in human therapy and its safety established for the purposes above.

Proteolysis of the IgG hinge exposes neo-epitopes that are targeted by host antibodies, termed human anti-hinge (“HAH”) antibodies. Further, IdeS cleavage in the lower IgG1 hinge exposes a neoepitope, -PAPELLG*GPSV (SEQ ID NO:10) (Ref. 2), to which abundant human autoantibodies bind. In fact, autoantibody binding to the IdeS cleavage site is of the highest magnitude observed among known IgG-degrading proteases (Ref. 9). The binding of human anti-hinge Abs to IdeS-cleaved mAbs paradoxically restored ADCC and CDC effector functions—an outcome that was unexpected since IdeS cleavage largely inactivates the IgG molecule itself. Further, serum anti-hinge IgGs were comprised disproportionately of the minor IgG3 subclass (normally only about 4% of total serum IgGs). IgG3 is particularly potent with regard to ADCC (Ref. 14).

Speculations regarding supplying IgG3 anti-hinge mAbs for enhanced ADCC immediately encounter a major obstacle. Namely, IgG3s possess the shortest circulating half-life of the IgG subclasses, are inherently unstable, and are subject to aggregation in a purified state (Ref. 24). These factors seriously detract from the appeal of developing an IgG3 anti-hinge therapeutic.

Polyclonal serum anti-hinge autoantibodies that were affinity-purified on F(ab′)2 fragments were shown to restore lost killing actions to IgG1 F(ab′)2s on MDA MB21 cancer cells (Ref. 9). Similarly-purified serum anti-hinge preparations restored a degree of in vitro ADCC function to single-cleaved pertuzumab (a humanized monoclonal IgG antibody capable of binding HER2 receptors) on SKOV3 cancer cells (Ref. 19). These findings were based on the use of purified systems in vitro and were tested on cells in the absence of other serum components. The latter is a major disconnect with the in vivo environment. Nevertheless, the findings did provide a degree of confirmation that serum isolates could restore target cell killing to a protease-generated F(ab′)2 and scIgG fragments and supported the conjecture that IgG3 possesses effective Fc-mediated functions (Ref. 19).

A related animal investigation demonstrated that anti-hinge antibody titers could be purposely induced. Specifically, this was obtained with a peptide analog immunization using the putative Staphylococcus aureus (“S. aureus”) GluV8 protease cleavage site in rabbit IgG. This immunization regimen (in this case with a CSKPTSPPPE (SEQ ID NO:11) peptide) induced substantial anti-peptide titers and resulted in bactericidal action against S. aureus in the model (Ref. 10). This finding indicated that host anti-hinge antibody binding to GluV8-mediated cleavage products of host (rabbit) IgG on the bacteria had initiated cell killing pathways. The latter study provided confirmatory evidence (in addition to the platelet studies mentioned herein) that endogenous circulating Abs could effectively bring about cell clearance.

As a tool to examine the anti-hinge concept in greater detail, a recombinant human/rabbit chimeric IgG1 monoclonal antibody against the IdeS cleavage epitope in human IgG1 was engineered (mAb 2095-2) (Ref. 11). This mAb provided, in several in vitro cell-based systems, degrees of restoration of cell-killing functions to IdeS-cleaved, anti-cell surface mAb F(ab′)2s including anti-tissue factor, anti-CD20 and a surface TNF variant. In addition, the combination of an IdeS-cleaved version of a monoclonal anti-GPIIb/IIIa antibody plus a monoclonal anti-hinge antibody restored the missing functionality of the F(ab′) 2 fragment and also achieved time- and dose-dependent platelet clearance in dogs to degrees that exceeded the capacities of the uncleaved anti-platelet IgG (Ref. 11). A similar demonstration was made in a rat model at F(ab′)2 fragment doses at which the intact 7E3 IgG was essentially non-functional (Ref. 11). However, these were based on platelet models, and cannot be extrapolated to cancer therapy.

### BRIEF SUMMARY OF THE INVENTION

The present invention relates to methods of treating solid tumors and other anomalous proliferative cells that involves an unexpected and surprising interdisciplinary connection between molecular immunology, infectious disease and cancer treatment. In some embodiments, the claimed methods comprise the use of proteolytic enzyme from the S. pyogenes bacterium (IdeS) to disable IgG Fc-mediated effector functions through the cleavage of a single peptide bond in the IgG hinge, which results in the exposure of a cleavage site antigen in the IgG hinge domain to which anti-hinge antibodies can attach. The binding of HAH antibodies to the cleaved hinge restores the lost Fc-mediated effector functions and cell killing by providing an intact, Fc domain to serve as a surrogate for the “damaged” original to propel subsequent immune engagements (e.g. by NK cells). Together, these elements merge into a unique and novel pathway of potentiated cancer cell eradication. Indeed, a link between endogenous IgG3 HAH antibodies and immunotherapeutic cancer treatment (via IdeS-mediated neoepitope generation in IgG) was unpredictable and unexpected in view of the published literature.

In one embodiment, the inventive methods described herein rely on the exposure of a neo-antigen in the IgG hinge leading to immune recognition by IgGs present in circulation (most notably the potent IgG3 component) in healthy humans. The ability to exploit widespread, underlying IgG3 humoral immune functions for therapeutic anti-cancer uses is unexpected. In contrast, preclinical explorations in animal systems relied exclusively on recombinant monoclonal IgG1 anti-hinge constructs.

IdeS is not an expected or normal entity in tumor environments. Absent a systemic infection and localized bacterial incursion, IdeS release and IgG degradation would be limited to the sites of S. pyogenes infection (usually in the skin). While natural S. pyogenes infections may circulate IdeS to tumor sites in occasional circumstances, as noted by Coley (Ref. 1), this delivery was highly unpredictable. Thus, the present invention relies on the targeted delivery of IdeS-cleaved IgG monoclonal or polyclonal antibodies (or, by extrapolation, IdeS itself), to tumor sites to thereby provide cleaved anti-tumor antibodies for engagement with anti-hinge autoimmunity.

In some embodiments, disclosed here is a method of treating a condition in a subject in need thereof, the method comprising administering to the subject a therapeutically effective amount of a modified IgG antibody, said modified IgG antibody comprising an epitope having at least 80% sequence similarity to a peptide having an amino acid sequence PAPELLG (SEQ ID NO: 6). In the majority of applications, intravenous administration is preferred although localized, intra-tumoral injections can also be envisioned.

In some embodiments, a modified IgG antibody is generated by digesting an intact IgG antibody with an IdeS enzyme. In one aspect, the IdeS enzyme is a purified enzyme derived from S. pyogenes. In another aspect, the IdeS enzyme is a recombinant enzyme having at least 90%, or at least 95%, or 100% sequence similarity to native IdeS from S. pyogenes. In another aspect, the modified IgG antibody having the neo-epitope is generated by incubating an intact IgG mAb with IdeS. In some embodiments, incubating is conducted for an amount of time and under conditions sufficient for IdeS proteolysis of the intact IgG to provide the anti-cancer monoclonal antibody comprising an IdeS-generated neo-epitope. In some embodiments, the incubating is conducted at 37° C. for about 10 min to about 24 hours at a ratio of intact IgG to IdeS of about 1 to about 0.01 w/w to about 1 to about 0.001 w/w.

In some embodiments, the anti-cancer monoclonal antibody comprising an IdeS-generated neo-epitope comprises single cleaved IgG, F(ab′)2, or a mixture thereof. In some embodiments, the anti-cancer monoclonal antibody comprising an IdeS-generated neo-epitope is substantially free of intact IgG.

In some embodiments, the modified IgG antibody is genetically engineered to contain the neo-epitope, or the modified IgG antibody is chemically modified to contain the neo-epitope selected from the group consisting of SEQ ID Nos 1-9.

In some embodiments, an intact and unconjugated IgG containing the human Fc domain and human hinge is selected from the group of mAbs that target elements on the surface of human cancer cells for the specific purpose of cell killing or eradication. Relevant members of the currently FDA-approved group include those in Table 1 below as adapted from Reference 20. The list should not be read as to exclude biosimilar versions of the listed mAbs or to exclude similar-functioning anti-cancer mAbs under development. To the latter point, the list does not include antibody-drug conjugates, checkpoint inhibitory mAbs, bispecific mAbs, mAbs that target soluble growth factors and other soluble signaling entities.

TABLE 1
Exemplary FDA-approved mAbs suitable
for use with the present invention.

| Antibody | Antigen | Format | Indications |
|---|---|---|---|
| Cetuximab | EGFR | Chimeric IgG1 | Colorectal cancer, |
| | | | head and neck |
| | | | squamous cell |
| | | | carcinoma |
| Daratumumab | CD38 | Human IgG1 | Multiple myeloma |
| Dinutuximab | GD2 | Chimeric IgG1 | Neuroblastoma |
| Elotuzumab | SLAMF7 | Humanized IgG1 | Multiple myeloma |
| Isatuximab | CD38 | Chimeric IgG1 | Multiple myeloma |
| Mogamulizumab | CCR4 | Humanized IgG1 | Cutaneous T-cell |
| | | | lymphoma |
| Necitimumab | EGFR | Human IgG1 | Non-small cell lung |
| | | | cancer |
| Ofatumumab | CD20 | Human IgG1 | Chronic lymphocytic |
| | | | leukemia |
| Olaratumumab | PDGFR- | Human IgG1 | sarcoma |
| | alpha | | |
| Pertuzumab | HER2 | Humanized IgG1 | Breast cancer |
| Ramucirumab | VEGFR2 | Human IgG1 | Gastric cancer |
| Rituximab | CD20 | Chimeric IgG1 | B-cell lymphoma |
| Trastuzumab | HER2 | Humanized IgG1 | Breast cancer |

In some embodiments, the present invention is directed to a composition comprising a mAb that includes an amino acid sequence selected from SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, and combinations thereof. In some embodiments, the mAb that includes the amino acid sequence selected from SEQ ID NOs. 1-9 and combinations thereof is a modified form of an FDA-approved mAb selected from Cetuximab, Daratumumab, Dinutuximab, Elotuzumab, Isatuximab, Mogamulizumab, Necitimumab, Ofatumumab, Olaratumumab, Pertuzumab, Ramucirumab, Rituximab, Trastuzumab, and combinations thereof. In some embodiments, the amino acid sequence selected from SEQ ID NOs. 1-9 and combinations thereof is present on a surface of the mAb. As used herein the term “on a surface of the mAb” means that the amino acid sequence is at least partially accessible for interactions with other moieties, which can include being positioned at least partially in a pocket or cleft. In some embodiments, the amino acid sequence selected from SEQ ID NOs. 1-9 and combinations thereof is present on a surface of the mAb in at least one location, at least one to 100 locations, at least one to 50 locations, at least one to 30 locations, or at least one to ten locations.

The amino acid sequence selected from SEQ ID NOs. 1-9 and combinations thereof can be formed on a surface of a mAb by the methods disclosed herein, or any other known methods capable of forming an amino acid sequence on a mAb. Thus, the present compositions can be prepared by exposing a known, commercially available mAb to an IdeS protease to provide a mAb comprising an amino acid sequence selected from SEQ ID NOs. 1-9 and combinations thereof. In some embodiments, the resulting mAb comprising an amino acid sequence selected from SEQ ID NOs. 1-9 and combinations thereof is further isolated and/or purified.

In some embodiments, the inventive compositions described herein include a mAb comprising an amino acid sequence selected from SEQ ID NOs. 1-9 and combinations thereof in a sterile solution suitable for intravenous administration and/or injection. The compositions can optionally include a preservative (e.g., benzyl alcohol, phenol, a paraben, methylparaben, phenoxyethanol, a diol, and the like), a stabilizer, an isotonicity agent (e.g., NaCl, KCl, glycerin, a mono-saccharide such as dextrose, a di-saccharide, and the like), a buffer (e.g, acetate, phosphate, citrate, lactate, glutamate, and the like).

The compositions of the present invention and associated methods can further include storing a composition comprising a mAb that includes an amino acid sequence selected from SEQ ID NOs. 1-9 and combinations thereof in a glass, plastic, or other container prior to administration for a period of about 1 minute to about 24 months. Thus, the present methods and compositions comprise preparing an inventive mAb for immediate use, as well as storing the inventive mAb for extended periods of time prior to use.

In some embodiments, the intact IgG is an anti-CD20 mAb (e.g., rituximab) and the efficacy of in vitro cancer cell killing is quantified by a fluorescence-activated cell sorting (FACS)-based detection that employs a fluorophore label within the target cell. Cell killing is quantified as the disappearance of live, intact cells including circulating B-cells.

In some embodiments, the subject in need thereof is a human.

In some embodiments, a subject in need thereof for treatment with the compositions and/or methods of the present invention is a human who has previously received treatment for a cancer. For example, in some embodiments a subject in need thereof has previously been administered first-line chemotherapy, fludarabine and cyclophosphamide (FC), methotrexate, an anthracycline-based chemotherapy regimen, a TNF agonist therapy, melphalan, cyclophosphamide, doxorubicin, vincristine and prednisone (CHOP), and/or a mAb therapy, with or without co-administration of a secondary therapeutic agent.

In some embodiments, the present invention is also directed to a method of treating cancer in a subject in need thereof, the method comprising administering IdeS at or around a tumor in the subject in an amount sufficient to generate single chain IgGs on host IgGs already directed against the tumor. In some embodiments, such method further comprises pre-administering an anti-cancer monoclonal antibody to the subject in need thereof. In some embodiments, such method further comprises administering intravenous IgG preparations (“IVIg”) as a source of anti-hinge antibodies. IVIg is typically derived from pools of thousands of donors.

In some embodiments, the present invention is directed to enhancing endogenous anti-hinge immunity to the IdeS cleavage point in a subject in need thereof, the method comprising prior administration of an effective amount of a peptide neo-epitope analog comprising the terminal sequence PAPELLG (SEQ ID NO:6), or APELLG (SEQ ID NO:7), or PELLG (SEQ ID NO:8), or ELLG (SEQ ID NO:9) to the subject. In some embodiments the peptide neoepitope analog is 4 to 12 amino acids in length, or 5 to 12 amino acids in length, or 6 to 12 amino acids in length, or 7 to 12 amino acids in length, or 8 to 12 amino acids in length, or 9 to 12 amino acids in length, or 10 to 12 amino acids in length, or 11 to 12 amino acids in length. In some embodiments the sequence of the peptide neoepitope analog comprises an amino acid sequence selected from:

(SEQ ID NO: 1)
CDKTHPAPELLG
(SEQ ID NO: 2)
DKTHPAPELLG
(SEQ ID NO: 3)
KTHPAPELLG
(SEQ ID NO: 4)
THPAPELLG
(SEQ ID NO: 5)
HPAPELLG
(SEQ ID NO: 6)
PAPELLG
(SEQ ID NO: 7)
APELLG
(SEQ ID NO: 8)
PELLG
(SEQ ID NO: 9)
ELLG

and combinations thereof.

In some embodiments the peptide neoepitope analog includes an amino acid, including cysteine, for linkage purposes to a mAb.

In some embodiments, the methods of the present invention further comprise delivering a peptide neo-epitope analog comprising the terminal sequence PAPELLG (SEQ ID NO: 6) to the subject between 10 days and 3 weeks prior to administering the anti-cancer monoclonal antibody comprising an IdeS-generated neo-epitope. This immunization strategy enhances normal anti-hinge reactivity with particular benefits for those individuals at the low end of the scale of ELISA reactivity (FIGS. 1A-1B).

In some embodiments, the methods of the present invention further comprise delivering a peptide neo-epitope analog comprising the terminal sequence PAPELLG (SEQ ID NO: 6) to the subject 10 days to 3 weeks prior to administering IdeS.

In some embodiments, the peptide neo-epitope analog is delivered by direct administration. In further embodiments, the peptide neo-epitope analog is coupled to or administered with an adjuvant agent prior to delivery. In some embodiments, the peptide neo-epitope analog is delivered by administering an RNA-containing vector that contains and expresses the sequence of the peptide neo-epitope analog. In some embodiments, the peptide neo-epitope analog is delivered by administering a DNA-containing preparation encompassing the sequence of the peptide neo-epitope analog. In further embodiments, the peptide neo-epitope analog is delivered by administering an adenoviral vector that contains and expresses the sequence of the peptide neo-epitope analog.

In some embodiments, the IdeS-generated neo-epitope is generated by treating an intact IgG with IdeS. In some embodiments, the incubating is conducted for an amount of time and under conditions sufficient for IdeS proteolysis of the intact IgG to provide the monoclonal antibody comprising an IdeS-generated neo-epitope. In some embodiments, the incubating is conducted at 37° C. for about 10 minutes to about 24 hours at a ratio of [intact IgG] to [IdeS] of about 1 to about 0.001 w/w to about 1 to about 0.05 w/w, or about 1:0.02.

In some embodiments, the modified IgG antibody is administered to the subject along with the IdeS after the digestion by IdeS is complete, without first removing the IdeS.

In some embodiments, a method disclosed for treating cancer in a subject in need thereof, the method comprising co-administering a therapeutically effective amount of IdeS and a therapeutically effective amount of an IgG antibody in the subject, wherein the therapeutically effective amount of IdeS digests the IgG antibody or antibodies on the cancer cell surface to generate a modified IgG antibody comprising an IdeS-generated neo-epitope to which endogenous anti-hinge antibodies bind. In one aspect, the IgG antibody binds to a cancer specific antigen in the cancer cells. In another aspect, the binding of the anti-hinge antibodies to the modified IgG antibody helps enhance antibody-mediated killing of the cancer cells.

In some embodiments, a method is disclosed for treating cancer in a subject in need thereof, the method comprising administering a therapeutically effective amount of IdeS at or near a solid tumor site, wherein the IdeS digests endogenous anti-cancer autoantibodies at or near the solid tumor site to generate neo-epitope in the anti-cancer autoantibodies. The exposed neo-epitope then helps attract endogenous anti-hinge antibodies, thereby enhancing antibody-dependent cellular cytotoxicity (ADCC) against the solid tumor.

In some embodiments, the modified monoclonal antibody incorporating an IdeS-generated neo-epitope comprises single cleaved IgG. In some embodiments, the monoclonal antibody comprising an IdeS-generated neo-epitope is substantially free of intact IgG. For purpose of this disclosure, intact IgG means an IgG that has not been digested by IdeS on either chain.

### BRIEF DESCRIPTION OF THE FIGURES AND DRAWINGS

FIGS. 1A-1B provide graphic representations of the detection of binding of serum IgG3 autoantibodies to the intact version and the IdeS-cleaved F(ab′)2 version of two different human IgG1 mAbs. FIG. 1A depicts the results with rituximab (anti-CD20) and FIG. 1B shows the results with the anti-TNF mAb, infliximab. The x-axis specifies the respective versions of the mAbs (intact or fragmented). Results from 20 individual healthy donors at a 1:50 dilution are shown. Symbols depict the average ELISA signal obtained in three replicate analyses. The ELISA method is fully described in Ref. 9, and the present depiction is adapted from that presented in Ref. 9.

FIG. 2 provides a graphic representation of the in vitro effects of rituximab-pvvr (tradename Ruxience®)+/−IdeS following their respective pretreatment on Ramos cells (a human cancer line) using endogenous human IgG (pooled IVIg concentrate) as the source of anti-hinge antibodies. Cell killing was achieved by human natural killer cells, and was quantified using an assay based on a flow cytometric analysis of cell viability.

FIG. 3 provides a graphic representation of in vitro ADCC using cetuximab+/−IdeS treatment and human A431 cancer cells. CSFE-labeled A431 cells were treated in a similar manner to that in FIG. 2 but in this case using cetuximab and A431 cells. As detailed in the Examples, NK cells were added at an effector-to-tumor ratio of 5:1 and incubated for 1.25 hours at 37° C. in a CO₂ incubator. The tumor cell killing curve was obtained by analyzing the results by flow cytometry. The results provided in FIG. 3 provide an antibody dose-response killing curve with each point representing the mean tumor cell lysis.

FIG. 4A provides a graphic representation of in vivo B-cell clearance in monkeys. The experiment examined the effectiveness of Rituximab and single-cleaved Rituximab resulting from IdeS treatment in Cynomolgus monkeys. Two groups of 3 male monkeys each received a low IV dose (0.050 mg/kg) of Rituximab or the single-cleaved version. Blood was collected at post-dose time intervals and processed for flow cytometry to analyze the number of CD19-positive cells. The Rituximab (IdeS) represents the single-clipped IgG (scIgG), while the intact IgG is identified as Rituximab. The mean B cell clearance and SEM are indicated for each point compared to the 2-hour determination and a P value of less than 0.05 (denoted with *) shows statistical significance in the unpaired T-test.

FIG. 4B depicts the results of FIG. 4A in bar graph format.

FIG. 5 is a graphic representation of data showing that IVIg treatment after IdeS cleavage can restore/improve IgG effector function. As discussed in detail in Example 4, CSFE-labeled SKBR3 cells were exposed to IVIg at 5 mg/mL or culture media and incubated for an hour in a CO₂ incubator at 37° C. After the hour, the cells were washed, suspended in culture media (RPMI-10% FBS), and then exposed to IdeS (at a 1:150 dilution, μg: unit) or culture media for another hour. Following this step, the cells were washed again and incubated with IVIG (at 5 mg/mL) or culture media for another hour. At this point, NK cells were added to the tumor cells at a ratio of 5:1 and incubated for 1.25 hours. All incubations were carried out at 37° C. in a CO₂ incubator. The cells were then assessed for viability using flow cytometry, and the percentage of tumor cell lysis was calculated. Data is represented as the mean % Tumor cell lysis+/−SEM.

### DETAILED DESCRIPTION

Before the present methods and related compositions are described, it is to be understood that the inventions described and claimed herein are not limited to the particular processes, compositions, or methodologies described, as these may vary. It is also to be understood that the terminology used in the description is for the purpose of describing the particular versions or embodiments only and is not intended to limit the scope of the present inventions, which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the present invention, the preferred methods, devices, and materials are now described. All patents, patent applications, and other publications cited or otherwise mentioned herein are incorporated by reference in their entirety. Nothing herein is to be construed as an admission that the inventions as recited in the appended claims are not entitled to antedate such disclosure(s) by virtue of prior invention.

As used herein and in the appended claims, the use of “a,” “an,” and/or “the” is intended to include both the singular and plural (e.g., “one or more”) unless the context clearly dictates otherwise. Thus, for example, reference to a “cell” is a reference to one or more cells and equivalents thereof known to those skilled in the art, and so forth.

Unless specified, “%” may refer to a percent by weight percent, or a percent by volume, or a percent weight by unit volume, and the relevant units would be immediately apparent to one of ordinary skill in the art based on the context.

As used herein and unless otherwise defined, “cancer” refers to the growth, division or proliferation of abnormal cells in the body, and is synonymous with “proliferative disorder.” Cancers that can be treated with the methods and the compositions described herein include, but are not limited to, breast, head and neck, prostate, lung, pancreatic, kidney, liver, etc.

As used herein, and unless otherwise defined, the terms “treat,” “treating” and “treatment” include the eradication, removal, modification, management or control of a tumor or primary, regional, or metastatic cancer cells or tissue and the minimization or delay of the spread of cancer.

As used herein, and unless otherwise defined, the term “subject in need thereof” refers to an animal, including but not limited to a human or a non-human primate. In certain embodiments, the subject in need thereof is a human. In a particular embodiment, the subject in need thereof has or is susceptible to having (e.g., through genetic or environmental factors) cancer. In a further embodiment, the subject in need thereof has or is susceptible to having (e.g., through genetic or environmental factors) a tumor.

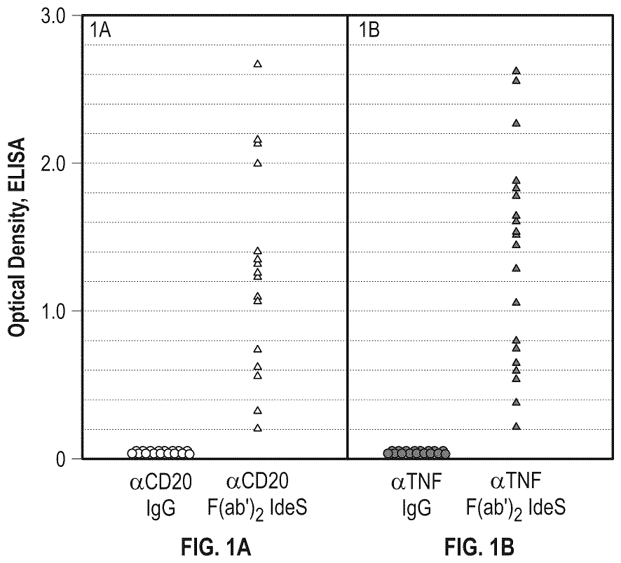

The methods of the present invention exploit for the first time existing, circulating anti-hinge antibodies—especially IgG3s—for therapeutic benefit. An important connection between the IdeS-generated neoepitope in individual anti-cancer antibodies to host anti-hinge antibodies that restore function to the cleaved IgGs is the preponderance of IgG3s in the group (FIG. 1). As discussed herein, IgG3s are normally a minor component of circulating IgGs (˜4%) yet possess among the highest capacity to mediate ADCC- and complement-mediated cell-killing. The overrepresentation of IgG3 in anti-hinge autoantibodies suggests a potential for its use in therapy. However, the IgG3 subclass is known to be unstable in purified form and has been a challenge to produce as a therapeutic. The present linkage between IdeS and natural IgG3 anti-hinge autoantibodies thus exploits a pathway not foreseen in the cancer literature.

The methods of the present invention function by linking abundant humoral autoimmunity against an IdeS-related neo-epitope that will be introduced onto an anti-cancer mAb. To be described, the method may be extended to the introduction of IdeS to an existing tumor environment such that the protease generates the neoepitope on already-bound anti-tumor antibodies (such Abs are known to be present in healthy blood but are often ineffectual as evidenced if by nothing more than the persistence of cancer in many individuals in the population). The resulting amplification in the efficacy of anti-cancer antibodies thereby results from the paradoxical induction of proteolytic “damage” to the anti-cancer mAb. Further, the methods of the present invention exploit an existing autoimmune repertoire against protease-generated IgG hinge epitopes. In some embodiments, the methods exploit the highly efficacious IgG3 component of humoral immunity.

In some embodiments, the methods of the present invention involve introduction of a cysteine proteinase, particularly IdeS as expressed in S. pyogenes, directly to a tumor site. Not being bound by any particular theory, local generation of tumor-bound scIgGs of the host will elicit the recognition of host IgG3s, thereby recruiting the IgG3 human anti-hinge (“HAH”) cohort.

Suitable injection sites include, but are not limited to, the head and/or neck. Other cancers suitable for treatment in this manner include lung cancers, breast cancers and highly localized cancers (e.g. prostate, pancreas, kidney, ovarian, brain) where surgical removal of the tumor is not always desirable or possible. A current compilation of anti-cancer cell mAbs have been defined that are suitable for use with the present invention: These have been tabulated above.

TABLE 2
Additional exemplary human anti-cancer mAbs currently in development
that can be used with the methods of the present invention.

| | | | | Major | |
|---|---|---|---|---|---|
| Name | Class | Target | Expected Indication | mechanism | Status |
| elotuzumab | Humanized IgG1 | CS1 | Multiple Myeloma | ADCC12, 16 | II/III |
| farletuzumab | Humanized IgG1 | Folate | Ovarian Cancer | | III |
| | | Receptor α | | | |
| necitumumab | Human IgG1 | EGFR | Non-Small Cell Lung | | III |
| | | | Cancer (NSCLC) | | |
| onartuzumab | Humanized IgG1 | c-Met | NSCLC/Gastric | | III |
| | | | Cancer | | |

Suitable therapeutic doses include the following injectable administration ranges for the readily available, high purity, recombinant IdeS for tumor sites are proposed: from 1 μg to 1 mg, and preferably from 5 μg to 500 μg. The present methods differ from other recent therapeutic applications of IdeS whereby administration sought to remove the entire circulating IgG cohort in autoimmune disease (Ref. 7)-a very different goal than the present one.

In some embodiments, a therapeutically effective dose of a composition of the present invention is from about 10 mg/m² to about 1,000 mg/m², about 20 mg/m² to about 750 mg/m², about 30 mg/m² to about 600 mg/m² of a mAb comprising an amino acid sequence of SEQ ID NOs. 1-9 or a combination thereof.

An IdeS-generated epitope can be generated by incubating an intact IgG with IdeS at an IdeS/IgG w/w ratio of about 0.001 to 1, or about 0.01 to 1, or about 0.05 to 1 at 37° C. for about 10 minutes to 24 hours, or for about 30 minutes to 24 hours, or for about 60 minutes to 24 hours, or for about 2 hours to 24 hours, or for about 4 to 24 hours, or for about 8 to 24 hours, or for about 12 to 24 hours, or for about 16 to 24 hours, or for about 20 to 24 hours.

An IdeS-related epitope can be introduced into rituximab in one of several ways—the first by straightforward IdeS proteolysis of rituximab IgG. As also mentioned herein, an IgG1 such as rituximab can be incubated with IdeS at an IdeS/IgG1 w/w ratio of about 0.001 to 1, or about 0.01 to 1, or about 0.05 to 1 at 37° C. for 10 minutes to 24 hours, or for about 30 minutes to 24 hours, or for about 60 minutes to 24 hours, or for about 2 hours to 24 hours, or for about 4 to 24 hours, or for about 8 to 24 hours, or for about 12 to 24 hours, or for about 16 to 24 hours, or for about 20 to 24 hours. IdeS protease can be easily removed from the mixture by adsorption of the scIgG on protein A or similar strategies. The resulting product can also be administered immediately without further purification (since IdeS is such a minor component) followed by refrigeration at 4° C. for up to 24 hours, or frozen until use.

IdeS digestion products of rituximab may include both scIgG and F(ab′) 2. Both derivatives expose the same hinge neo-antigen(s) for autoantibody binding. One potential therapeutic advantage of scIgG in vivo (vs. a F(ab′)2 fragment) is its longer PK profile due to retained FcRn binding as shown in mice (Ref. 6). The longer PK of scIgG vs. F(ab′)2 [days vs. hours] confers a longer contact time and exposure to tumor targets.

In some embodiments, a composition of the present invention is administered according the methods disclosed herein as an intravenous infusion, an injection, a subcutaneous injection, or another known method of administering a mAb to a subject in need thereof.

The inventive methods can include a single administration of a composition as described herein, as well as dosage regimens that comprise multiple doses of the inventive compositions.

A “priming” approach prior to mAb scIgG therapy is the augmentation of endogenous anti-hinge immunity by immunization with the IdeS cleavage point peptide analog. This immunization tactic has multiple parallels in the current era (e.g. COVID-19). For the IdeS-induced hinge epitope, humans already widely recognize this antigen although to differing degrees, (as evident in the ranges of autoimmune IgG3 reactivity to IdeS-cleaved IgGs in FIG. 1). An immune supplementation approach to enhance endogenous anti-hinge immunity prior to treating with the mAb-neo-antigen conjugate would enhance the actions of anti-tumor mAbs as they get bound by endogenous anti-hinge autoimmunity. This same vaccination priming approach would also serve ahead of all the above-described cleavage-site antigen delivery approaches to the tumor sites. An alternative to the above active immune supplementation approach would be a passive administration of polyclonal anti-hinge antibodies present in commercial Intravenous IgG preparations (“IVIg”) as typically derived from pools of thousands of donors.

In some embodiments, IdeS is introduced to the tumor environment in order to locally generate scIgGs directed to the tumor. As background, host humoral immunity commonly generates some degree of endogenous anti-tumor immunity, including polyclonal and heterogeneous autoantibodies, but this is often insufficient to fully control tumor proliferation and metastasis. Such Abs might conceivably be subject to host protease cleavages by MMPs (Ref. 15), but less likely to an exogenous enzyme such as IdeS from S. pyogenes, which would function at the skin infection site rather than at an internal tumor site. Cell-bound mAbs were previously shown to be susceptible to MMP3 cleavage in vitro (Ref. 6) and especially to gluV8 cleavage in vivo (Ref. 10), and there is ample reason to expect that a similar phenomenon would apply to IdeS (Ref. 27).

Not being bound by any particular theory, this mechanism points to a potentially therapeutic conversion of existing cell-bound host anti-TAA antibodies in that local environment to scIgGs without a need to know or define what they actually bind to. This strategy exploits host immunity at two separate levels: 1) endogenously elicited anti-TAA Abs to be converted by IdeS to scIgG versions, and 2) endogenous anti-cleavage site Abs to increase the potency of the IdeS-generated scIgGs. A successful in vitro application of this pathways is shown in Example 4.

In some embodiments, IdeS is delivered directly to a tumor site in a subject in need thereof. Suitable delivery mechanisms include, but are not limited to, direct injection into the tumor site by needle, encompassing IdeS within or on nanoparticles, immune conjugates, as well as encapsulation within liposomes. Such approaches take advantage of the fact that cell-bound Abs are especially susceptible to local IdeS cleavage (Ref. 6 and 18).

Given the small amount of IdeS required to be delivered, it is expected that IdeS administered directly to a tumor site would primarily act locally and have minimal impact on circulating IgGs and humoral immunity in general (Ref. 7). The total administration level of purified IdeS is defined to be within the range of 10 μg to 500 μg.

Plasma/sera from human individuals (but not mice, rats, dogs) already provide varying levels of antibody reactivity to the PAPELLG*(SEQ ID NO:6) sequence. This is a substantial disadvantage for the deployment of preclinical anti-tumor models. Alternatively, monoclonal anti-hinge secondary mAb treatments can restore clearance to c7E3 IdeS-generated F(ab′)2-coated platelets in dogs and rats. However, as a therapeutic approach, it is not attractive for human therapy since it would require two successive mAb administrations.

The present disclosure is further illustrated by the following items:
Item 1. A method of treating a condition in a subject in need thereof, the method comprising administering to the subject a therapeutically effective amount of a modified IgG antibody, said modified IgG antibody comprising an epitope having at least 80% sequence similarity to a peptide having an amino acid sequence selected from the group consisting of the epitope sequences of SEQ ID NOs: 1-9.
Item 2. The method of Item 1, wherein the condition treated is selected from the group consisting of: viral diseases, bacterial diseases, fungal diseases, parasitic diseases, and proliferative diseases other than cancer.
Item 3. The method of any preceding Items, wherein the condition treated is cancer.
Item 4. The method of any preceding Items, wherein the epitope comprises the sequence: PAPELLG (SEQ ID NO: 6) with a free C-terminus.
Item 5. The method of any preceding Items, wherein the modified IgG antibody is generated by digesting an intact IgG antibody with an IdeS enzyme.
Item 6. The method of any preceding Items, wherein the modified IgG antibody is genetically engineered to contain the epitope or wherein the modified IgG antibody is chemically modified to contain the epitope.
Item 7. The method of any preceding Items, wherein the digestion is conducted for an amount of time and under conditions sufficient for IdeS proteolysis of the intact IgG to provide the IgG antibody comprising an IdeS-generated epitope.
Item 8. The method of any preceding Items, wherein the digestion is conducted at 37° C. for about 10 minutes to about 24 hours wherein ratio of [intact IgG] to [IdeS] is between 1 to 0.001 w/w and 1 to 0.05 w/w, or about 1:0.02.
Item 9. The method of any preceding Items, wherein the epitope is a neo-epitope to the subject.
Item 10. The method of any preceding Items, wherein the modified IgG antibody is administered to the subject along with the IdeS enzyme.
Item 11. The method of any preceding Items, wherein the IdeS enzyme is a recombinant IdeS.
Item 12. The method of any preceding Items, wherein the intact IgG antibody is selected from the group consisting of: cetuximab, daratumumab, dinutuximab, elotuzumab, isatuximab, mogamulizumab, necitimumab, ofatumumab, olaratumumab, pertuzumab, ramucinumab, rituximab, trastuzumab and generic versions and or combinations thereof.
Item 13. The method of any preceding Items, wherein the intact IgG antibody is selected from the group consisting of: anti-viral antibodies, anti-bacterial antibodies, anti-fungal antibodies, anti-parasitic antibodies.
Item 14. The method of any preceding Items, wherein the subject in need thereof is a human.
Item 15. The method of any preceding Items, wherein the subject comprises an endogenous anti-hinge antibody.
Item 16. The method of any preceding Items, wherein the modified IgG antibody is a single-cleaved IgG (“scIgG”).
Item 17. A method of treating cancer in a subject in need thereof, the method comprising co-administering a therapeutically effective amount of IdeS and a therapeutically effective amount of an IgG antibody in the subject, wherein the therapeutically effective amount of IdeS digests the IgG antibody or antibodies on the cancer cell surface to generate a modified IgG antibody comprising an IdeS-generated neo-epitope to which endogenous anti-hinge antibodies bind.
Item 18. The method of Item 17, wherein the IgG antibody binds to a cancer specific antigen.
Item 19. A method for treating cancer in a subject in need thereof, the method comprising administering a therapeutically effective amount of IdeS at a solid tumor site, wherein the IdeS digests endogenous anti-cancer autoantibodies at the solid tumor site to generate neo-epitope in the anti-cancer autoantibodies which attract endogenous anti-hinge antibodies, thereby enhancing antibody-dependent cellular cytotoxicity (ADCC) against the solid tumor.
Item 20. The method of any of Item 19, wherein the subject in need thereof is a human.
Item 21. The method of any of Items 1-16, wherein the activity of the IgG antibody comprising the Ides-generated epitope is 5 to 500 fold greater in vitro than an IgG antibody not treated with IdeS.

### EXAMPLES

### Example 1

The present example describes the binding of IgG3 autoantibodies to intact and IdeS-cleaved rituximab (anti-CD20 mAb) (FIG. 1A, Table 3) and intact and IdeS-cleaved anti-tumor necrosis factor (anti-TNF) mAb (FIG. 1B, Table 3), as detected by a commonly employed ELISA (Ref. 9).

ELISA: the assay employed the parental IgG or protease-derived F(ab′)2 fragments coated on 96-well plates at 10 μg/mL. Individual human serum samples collected from 20 healthy subjects were incubated in the wells at 1:50 dilution. Detection of bound IgG3 antibodies was carried out with an HRP-conjugated anti-human IgG3 mAb (Zymed, 1:300 dilution). The plates were developed using SIGMAFAST OPD (Sigma-Aldrich) and stopped with acidification with HCl. Expanded details are given in Ref. 9.

Differential serum-derived IgG3 binding to the IdeS-cleaved IgGs compared to intact parental IgGs was shown in ELISA (Table 3). Individuals (n=20) were all reactive—some robustly so—to the IdeS-generated F(ab′)2. In contrast, there was little incidence of IgG3 recognition to the intact IgG1 counterparts. Serum provides IgG3 Abs that would be difficult to attempt with monoclonal IgG3 entities since stable, therapeutic IgG3 mAbs have proven notoriously difficult to produce. Instead, the pre-existing serum cohort of IgG3 anti-hinge antibodies (above) can be exploited for their own therapeutic efficacy at tumor sites. Results are provided in Table 3 and displayed graphically in FIGS. 1A-1B, respectively.

TABLE 3
Data from differential serum-derived IgG3 binding to the
IdeS-cleaved IgGs compared to intact parental IgGs.

| | | IdeS-cleaved | | IdeS-cleaved |
|---|---|---|---|---|
| | Intact mAb1 | mAb1 | Intact mAb2 | mAb2 |
| Optical | <0.02 | 0.28 | <0.02 | 0.2 |
| Density, | <0.02 | 0.42 | <0.02 | 0.3 |
| ELISA | <0.02 | 0.58 | <0.02 | 0.5 |
| | <0.02 | 0.62 | <0.02 | 0.55 |
| | <0.02 | 0.70 | <0.02 | 0.6 |
| | <0.02 | 0.75 | <0.02 | 1.0 |
| | <0.02 | 1.05 | <0.02 | 1.05 |
| | <0.02 | 1.30 | <0.02 | 1.25 |
| | <0.02 | 1.45 | <0.02 | 1.3 |
| | <0.02 | 1.50 | <0.02 | 1.35 |
| | <0.02 | 1.45 | <0.02 | 1.42 |
| | <0.02 | 1.58 | <0.02 | 2.0 |
| | <0.02 | 1.60 | <0.02 | 2.1 |
| | <0.02 | 1.78 | <0.02 | 2.25 |
| | <0.02 | 1.83 | <0.02 | 2.7 |
| | <0.02 | 1.85 | <0.02 | 0.7 |
| | <0.02 | 2.25 | <0.02 | 1.05 |
| | <0.02 | 2.4 | <0.02 | 1.15 |
| | 0.05 | 2.55 | 0.07 | 1.2 |
| | 0.15 | 2.6 | 0.20 | 1.4 |

Referring to FIGS. 1A-1B, symbols represent the average ELISA signal obtained for each serum sample in three replicate analyses. Individuals (n=20) were all reactive-some robustly so-to the IdeS-generated F(ab′)2. In contrast, there was little incidence of IgG3 recognition to the intact IgG1 counterparts. These results point to normal circulation providing natural IgG3s to serve as anti-hinge immune modulators.

The near-universal presence of human serum autoantibodies to the IdeS-generated epitope in the IgG hinge prompted attempts to define their possible function. Larger scale affinity purification of human serum autoantibodies to the IdeS-generated epitope in the IgG hinge for reagent uses was thwarted by their generally low concentration in some individual serum and even pooled IVIg (est. 5 ug/mL; ˜0.02% of total IgGs). A surrogate approach was adopted by development of a monoclonal rabbit/human anti-hinge antibody termed 2095-2. The 2095-2 mAb was used to target the IdeS-generated F(ab′)2 fragment of abciximab. Abciximab targets the GPIIb/IIIa receptor on human platelets and, at lower affinity, to the analogous receptor on dog and rat platelets, (Ref. 16). The cross-reactivity enabled animal models of platelet clearance to be devised. These animal studies unexpectedly revealed that the combined use of mAb 2095-2 together with abciximab F(ab′)2 resulted in more rapid decline and a greater extent of platelet count lowering than was possible with abciximab IgG alone. This was a key insight that the combined “sandwich” of anti-hinge and F(ab′) 2 achieved an amplified cell clearance effect. Further it was an important demonstration that the anti-hinge concept could operate in vivo.

Despite the importance of the platelet studies with monoclonal anti-hinge mAbs as proofs-of-concept for cell clearance, the relevance of these to cancer is limited. First, the canine and rat platelet studies were normal hematologic cell-counting tests and not tumor systems. Those demonstrations had relied on a monoclonal IgG1 anti-hinge antibody to target the abciximab F(ab′)2 fragment-not scIgG-on the animals' platelets. Thus, despite its value as a model, platelet clearance must be considered to be only a surrogate for cancer cell eradication. To pursue a more relevant line of inquiry, a human system using endogenous host antibodies as the anti-hinge component and scIgG as the adaptor to a cell was needed. A human cancer cell system was devised as described below.

### Example 2

Since one intended application of this therapeutic proposal is the treatment of human solid tumors, non-human primates were unfortunately not usable since monkey tumor models were not available. Thus, it was necessary to focus on a human in vitro ADCC system.

The ADCC protocol exposes anti-CD20 rituximab-coated cancer cells (+/−IdeS protease treatment) with pooled human IgG as a source of anti-hinge antibodies and followed by exposure to human natural killer cells to induce antibody-dependent cellular cytotoxicity. Ruxience® is a biosimilar version of rituximab known as rituximab-pvvr. The assay is described in Ref.13.

The materials for flow cytometric analysis of ADCC were as follows: Pooled human serum IgG (IVIg, Sigma-Aldrich) provided ample levels of endogenous anti-hinge antibodies. Purified human NK cells (obtained from STEMCELL technologies) were the immune effector killing cells and their use averted the need to frequently obtain fresh donor blood. The CD20-expressing cancer cell was the human Ramos cell line (ATCC CRL-01596, labeled with CFSE fluorophore; ThermoFisher). The targeting monoclonal was the anti-CD20 rituximab+/−treatment with IdeS (Promega®) for 24 hours at 37° C. using a 0.2% ratio of IdeS (2 ug/mL) to rituximab (1 mg/mL). The latter IdeS to IgG ratio had previously been shown to yield a majority of scIgG product as opposed to F(ab′)2 (Ref. 6).

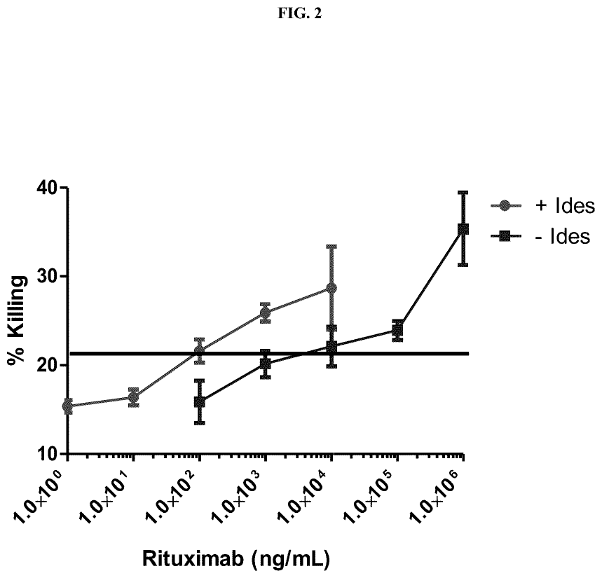

A biosimilar version of rituximab (i.e., Rituximab-pvvr, tradename Ruxience®) was investigated with Ramos cells to determine if anti-hinge antibodies present in IVIg could drive the killing of a cancer cell. CSFE-labeled Ramos cells were incubated with rituximab-pvvr treated with IdeS or with intact IgG. NK cells were added at a ratio of 5:1 (effector:tumor) and incubated in the presence of 5 mg/mL IVIg for 2 h at 37° C. in a CO₂ incubator. Cell killing was analyzed by flow cytometry (see Ref. 13), and the results are displayed graphically in FIG. 2. Referring to FIG. 2, the antibody dose-response killing curve results are shown with each point representing the mean±SEM.

A pilot experiment using a serial dilution series of IVIg demonstrated that IVIg 0.25 mg/mL would provide substantial killing to IdeS-treated rituximab added to cells at 1 μg/mL whereas this concentration of intact rituximab had no effect (not shown). IVIg at 0.25 mg/mL is low compared to normal IgG in circulation (˜10 mg/mL) so a subsequent test was carried out at fixed 5 mg/mL IVIg into which rituximab+/−IdeS was serially diluted and then exposed to the cells. FIG. 2 demonstrates that cell killing was consistently higher (Y-axis) with concentrations of IdeS-treated rituximab compared to equivalent concentrations of the parent antibody. More importantly, the concentrations of each required to achieve a comparable killing level (X-axis) were offset in the respective serial dilution curves. For example, a 20% level of killing occurred at 100 ng/mL for IdeS-cleaved rituximab compared to 10 ug/mL for intact. This off-set suggests a 100-fold differential in the concentration-effect outcome. Thus, this result showed a large functional advantage to a protease degradation product that would otherwise be expected to be dysfunctional compared to its intact parent. The non-obvious reason is the contribution of anti-hinge antibodies contained in IVIg.

The novelty of this demonstration is that healthy human blood (e.g., the IVIg isolate) provided the anti-hinge antibodies (as opposed to earlier reports using primarily monoclonal anti-hinge antibodies as a model system).

Not being bound by any particular theory, the mechanistic basis for this enhancement likely derives from an optimal presentation of the surrogate Fc of the anti-hinge antibody to NK cells than is inherent to native rituximab alone. The “sandwich” of anti-hinge Abs on cleaved rituximab can be envisioned to be positioned differently on the cell surface than is the primary IgG. In any case, it was the potentiating effect of IgGs from healthy circulation that was unexpected. This is the first time that human serum antibodies contribute effector function to a disabled anti TAA antibody to yield substantial amplification of its concentration effect relationship on an actual cancer cell.

Not being bound by any particular theory, the present results implicate the presentation of the IdeS-generated IgG epitope at the cancer surface so that endogenous host antibodies can bind to it. Indeed, there is little reason for such an IgG epitope to occur naturally in that location since the only source of IdeS is S. pyogenes and such bacterial infections are usually remote.

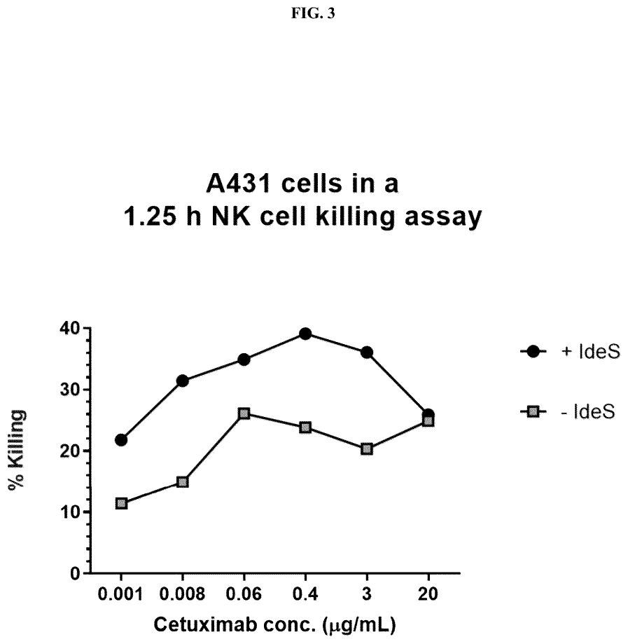

The implications of this invention extend beyond the particular mAb or antibody and cellular components of the present experiment. Rituximab has been prominently mentioned herein but other mAbs such as trastuzumab (herceptin; anti-EGFR) and pertuzumab (anti-HER2) are further candidates for investigation. For example, FIG. 3 depicts the comparative cell killing effects of IdeS-cleaved Cetuximab and its parent IgG (anti-EGFR) on a separate human cancer cell line, A431 cells. Again, IVIg provided the anti-hinge antibodies to facilitate NK-cell killing. Thus, the anti-hinge paradigm is not limited to a specific mAb of cancer cell line. A series of in vitro cell killing assays were conducted with mAbs and different cancer cell lines as tabulated in Table 4. Examples 1˜4 in Table 4 list the in vitro findings.

TABLE 4
IgGs from normal circulation provide anti-hinge reactivity
to restore cell-killing to IdeS-cleaved mAbs^{a}

| | | | | | | Relative |
|---|---|---|---|---|---|---|
| | Anti-cancer | | | | | efficacy |
| | mAb or other | | | Anti-hinge Ab | | vs. |
| | source | IdeS | | source | Cell | uncleaved |
| Ex. | (1° incubation) | product(s) | Cell target | (2° incubation) | killing | mAb^{k} |
| 1 | Rituximab^{b} | F(ab′)2 | Ramos | IVIg^{d} | Isolated | +++ |
| | | scIgG | cells^{c} | | human NK | |
| | | | | | cells | |
| 2 | Rituximab | scIgG/ | Daudi | IVIg | Isolated | + |
| | | F(ab′)2 | cells^{e} | | human NK | |
| | | | | | cells | |
| 3 | Cetuximab^{g} | scIgG/ | A431 | IVIg | Isolated | ++ |
| | | F(ab′)2 | cells^{h} | | human NK | |
| | | | | | cells | |
| 4 | Trastuzumabⁱ | scIgG/ | SKBR3 | IVIg | Isolated | + |
| | | F(ab′)2 | cells^{j} | | human NK | |
| | | | | | cells | |
| 5 | Rituximab | sclgG | Cyno. | Endogenous | Endogenous | ++ |
| | In vivo | | monkey | circulating | monkey | |
| | | | B-cells^{f} | antibodies | NK cells | |

^{a}In vitro ADCC assay using purified human NK cells unless otherwise indicated
^{b}Anti-CD20 human/murine chimeric mAb
^{c}Human B lymphocyte cells
^{d}Intravenous gamma globulin obtained from pooled healthy human donors
^{e}Human lymphoblast cancer cells
^{f}Circulating B-cells detected by FACS using the alternative CD-19 marker
^{g}mAb inhibitor of human epidermal growth factor receptor 2 (HER2)
^{h}Human epidermoid carcinoma cells
ⁱmAb inhibitor of human epidermal growth factor receptor 2 (HER2)
^{j}Human breast carcinoma cells
^{k}Relative efficacy as considered appropriate to individual assay systems, mAbs, and target cells

### Example 3

A variety of tumor models, including human cancers, have been devised in mice of various types. Unfortunately, few can be readily applied to the IdeS/anti-hinge Ab system described above using the human immune system. Mice and other non-primate species do not possess anti-hinge Abs comparable to humans and show limited susceptibility to S. pyogenes infection and IdeS cleavage of IgG. While non-human primates do demonstrate anti-hinge Abs to the IdeS cleavage site in IgG, no tumor models exist in these animals. For these reasons, a model of normal B-cell clearance in cynomolgus monkeys was undertaken. B-cells can be precursors to lymphoma cells in humans. Monkey B-cells express a related surface receptor to human CD-20 and mAbs that target this receptor have been shown to deplete B-cells in this species (Ref. 25). Thus, a comparative study of rituximab and its IdeS-cleaved derivative was devised for the clearance of circulating B-cells.

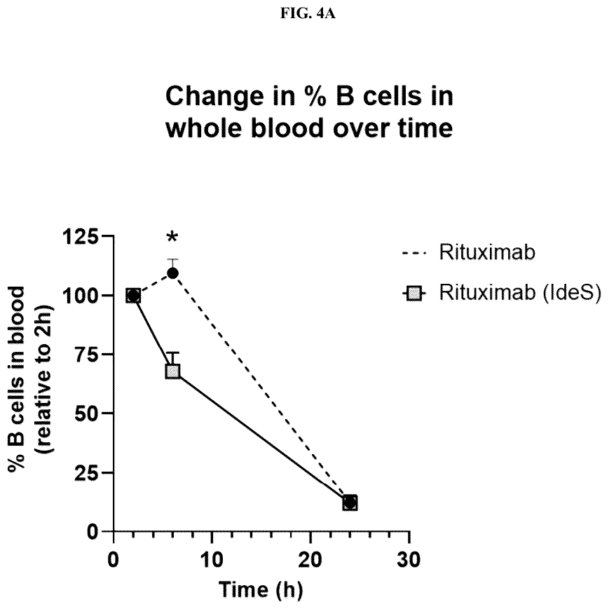

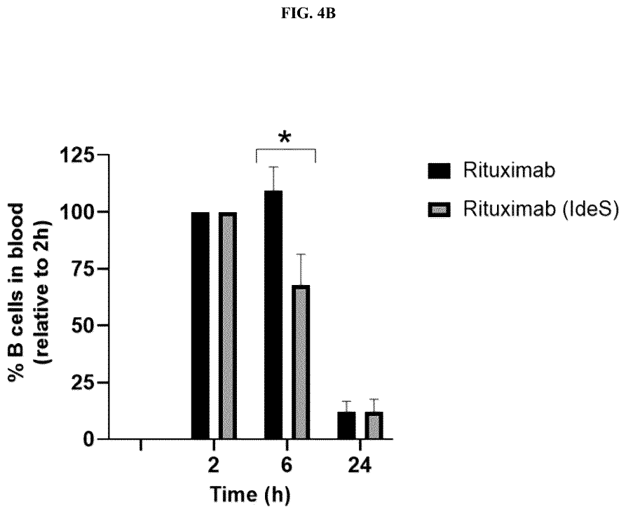

Cynomolgus macaque monkeys (3 per group) were administered intact rituximab or IdeS-treated rituximab at 0.05 mg/kg. The IdeS digestion conditions were designed to yield primarily scIgG rather than F(ab′)2 using a 1:2000 IdeS:IgG ratio (w/w) overnight at 37° C. The 0.05 mg/kg dose was predetermined to be adequate to induce a progressive 24-hour depletion by intact rituximab for comparison to the IdeS-cleavage group. B-cell counts were quantified at predose, at 2 hours, 6 hours and 24 hours post dose. Dosing and blood samplings were intravenous. Cell counts were established using flow cytometry and an anti-CD-19 probe to estimate circulating B-cell numbers. The 2-hour determinations were taken as the basis for the evaluation of time-dependent depletion thereafter due to pharmacokinetic changes that occur after administration (such as antibody binding and/or internalization) and the circulating mAb concentration can be assumed to have reached steady state by this time. Thus, each animal was normalized to its measured value at t=2 h and the results were quantified as the % B cells in whole blood over time. The results are tabulated in Table 5, Example 4, and depicted graphically in FIGS. 4A (line graph) and 4B (bar graph).

The results in FIGS. 4A-4B are notable for reasons that have been described in previous examples. In particular, the IdeS-treated rituximab which has been repeatedly shown to be dysfunctional when tested in in vitro ADCC assays, is here shown to be as active as intact rituximab at B cell clearance at 24 hours. Thus, the circulation of the monkeys provided the component to restore function to the scIgG that can be presumed to be anti-hinge Abs. Moreover, the results at six hours indicate a more rapid early decline than for intact rituximab. This echoes the amplification phenomenon seen previously. Also, this study is the first to employ a normal circulating cell with relevance to cancer (B cells are precursors to a type of lymphoma for which anti-CD20 mAb therapy is used in humans). Perhaps most importantly, this experiment provides critical in vivo confirmation for the various human in vitro findings detailed above.

### Example 4

Antibodies that target tumor cells have been derived from healthy normal humans (Ref. 26). Although such endogenous Abs are unlikely to be sufficient to contain tumor growth in all cases, we investigated whether IdeS treatment of tumor cells coated with such endogenous Abs could establish or amplify cell killing in the human in vitro ADCC system.

The following experimental design was employed. CSFE-labeled SKBR3 cells were exposed to IVIg at 5 mg/mL or culture media and incubated for an hour in a CO₂ incubator at 37° C. After the hour, the cells were washed, suspended in culture media (RPMI-10% FBS), and then exposed to IdeS (at a 1:150 dilution, μg: unit) or culture media for another hour. The IdeS protease treatment was performed with the intent of converting any cell bound IgGs to scIgG or F(ab′)2 derivatives. Following this step, the cells were washed again and incubated with IVIg (at 5 mg/mL) or culture media for another hour. The re-exposure to IVIg was conducted with the intent of providing anti-hinge Abs to any cleaved anti-tumor Abs on the cell surface. At this point, NK cells were added to the tumor cells at a ratio of 5:1 and incubated for 1.25 hours. All incubations were carried out at 37° C. in a CO₂ incubator. The cells were then assessed for viability using flow cytometry, and the percentage of tumor cell lysis was calculated. Cell killing was generated in the presence of human NK cells and the results were quantified using the FACS methods previously described. Results are presented in Table 5. Data is represented as the mean % Tumor cell lysis+/−SEM.

TABLE 5
Antibodies from normal circulation are sufficient
to provide both anti-cancer Abs and anti-IdeS
cleavage site Abs to amplify IgG cell-killing

| Target | Source | | Anti-hinge | | Relative |
|---|---|---|---|---|---|
| cancer | of anti- | | Ab source | ADCC | efficacy |
| cell | cancer | Protease | (secondary | effector | after IdeS |
| line | Abs | treatment | incubation) | cells | cleavage^{k} |
| SKBR3 | IVIg | IdeS on | IVIg | isolated | + |
| cells | | IVIg- coated | | human NK | |
| | | cells | | cells | |
| A431 | IVIg | IdeS on | IVIg | isolated | + |
| cell | | IVIg- coated | | human NK | |
| | | cells | | cells | |

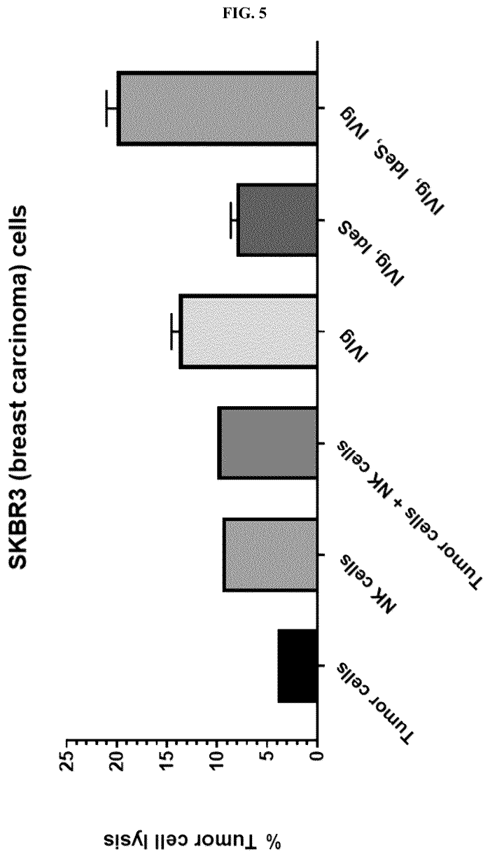

A graphic presentation of the results with SKBR3 cells and IVIg as the source of anti-cancer cell Abs is presented in FIG. 5. Referring to FIG. 5, a key comparison is the last two bars in which cells treated with IVIg and then with IdeS but without subsequent IVIg exposure (second most right-handed bar) shows a markedly lower level of cell killing than with cells that received a secondary exposure to IVIg. Thus, normal healthy human blood antibodies against tumor cells exist and can be substantially elevated in function by exposure to IdeS.

### Example 5 (Prophetic)

An anti-cancer mAb (e.g. herceptin) will be administered to a human subject suffering from breast cancer (or another EGFR-related cancers) after pre-exposure to IdeS. IdeS will be used catalytically (e.g., in a ratio of 0.001 to 0.1 w/w relative to the mAb) to generate scIgG. After exposure, the scIgG may be further purified, or may be used directly (without removal of remaining IdeS), since recombinant IdeS is approved for human use (e.g., Imlifidase).

The scIgG will be administered to a human subject by intratumoral delivery in an amount of 1 μg to 100 ug. Pretreatment of a mAb to scIgG does not decrease the circulating half-life since the Fc domain remains associated.

The measurement of anti-tumor outcomes will be conducted using standard techniques for this purpose. Such metrics include ultrasonography, MRI, CBCT (cone beam computed tomography) and manual methods involving diametric-based ellipsoid tumor volume measurement. The intent is to quantify the effects of respective treatments on tumor volumes over time.

### Example 6 (Prophetic)

The current findings also raise the possibility that the application of IdeS itself in the localized tumor environment could trigger the epitope generation on any bound (host) anti-TAA antibodies and thereby set a path of endogenous tumor regression in motion. By this antigenic generation on the cancer cell surface, IdeS will act to enhance the activity of host antibodies that themselves offer little or no protection against cancer cell proliferation.

Recombinant IdeS (e.g., Imlifidase) will be administered directly to a human tumor at a dose of 1 ug to 1 mg. Endogenous scIgG anti-tumor mAbs will be produced in situ by Abs present at the injection site(s).

Therapeutic efficacy will be determined as in Example 3.

### Example 7 (Prophetic)

A further example will demonstrate the use of IdeS “accelerant” in vivo. The anti-cancer effect of IdeS-mediated amplification of cleaved IgG functions depends on the presence of endogenous anti-hinge antibodies in individual patients. FIGS. 1A-1B show that virtually all individuals possess some antibody reactivity against the IdeS cleavage site epitope but that there is considerable variability among them.

Human serum anti-hinge reactivity can be increased by any of a number of immunization strategies that induces antibodies against the APELLG (SEQ ID NO:7) epitope. A peptide analog of the gluV8 cleavage site in rabbit IgG was successfully used as a KLH (keyhole limpet hemocynanin)-adduct to block S. aureus growth in a rabbit model (Ref. 10). Approaches for human use could also employ vaccination strategies using RNA, DNA, and adenoviral expression of the IdeS-generated epitope in IgG-parallel approaches to those recently used successfully for vaccine development against the spike protein of coronaviruses. The overriding concept is that once the IdeS-cleavage epitope is present on a tumor surface, the host anti-hinge antibodies will facilitate the immune mechanisms to eradicate the tumor.

The invention as a whole is not limited by any of the above examples and is not limited to any one cancer or solid tumor, but rather has application to other cellular pathogens such as, but not limited to, bacteria, viruses, and other invasive organisms (tissues) against which immune augmentation would aid the host in their destruction. Known anti-pathogen mAbs can be treated with IdeS to elicit the augmentation. Analogously from other examples, one could direct an infusion of IdeS into an active infection site to generate enhanced host antibodies against the pathogen. Again, the system relies on a host anti-hinge system that is human specific.

### REFERENCES

1. Hoption Cann S A et al. 2003 Dr William Coley and tumour regression: a place in history or in the future. Postrgrad Med J 79; 672-680.
2. von Powell Ramingen et al. 2002. IdeS, A novel streptococcal cysteine proteinase with unique specificity for immunoglobulin G. EMBO J. 21; 1605-1615.
3. Vincents B et al. 2004 Enzymatic Characterization of the Streptococcal Endopeptidase, IdeS, Reveals That It Is a Cysteine Protease with Strict Specificity for IgG Cleavage Due to Exosite Binding. Biochemistry, 49, 15540-15549
4. Dall'Acqua W et al. 1998 Contribution of Domain Interface Residues to the Stability of Antibody CH3 Domain Homodimers. Biochemistry 37, 9266-9273.
5. Zhang N. et al. 2015 Dysfunctional Antibodies in the Tumor Microenvironment Associate with Impaired Anti-cancer Immunity, Clin. Cancer Res. 21, 5380-5390.
6. Brezski, R. J. et al, 2009 Tumor-associated and microbial proteases compromise host IgG effector functions by a single cleavage proximal to the hinge. Proc. Nat. Acad. Sci. 106, 17864-17869.
7. Jordan S C et al. 2017, IgG Endopeptidase in Highly Sensitized Patients Undergoing Transplantation NEJM 377; 453.
8. Tazi I et al. 2011, Monoclonal antibodies in hematological malignancies; past, present and future, J. of Cancer Research and Therapeutics 7, 399-407.
9. Brezski, R J. et al. 2008. Human anti-IgG1 hinge autoantibodies reconstitute the effector function of proteolytically-inactivated IgGs. J. Immunol. 181, 3183-3192.
10. Jordan R E, et al. 2016 A peptide immunization approach to counteract a Staphylococcus aureus protease defense against host immunity. Immunology Lett. 172, 29-39.
11. Brezski R J et al 2014 A monoclonal antibody against hinge-cleaved IgG restores effector function to proteolytically-inactivated IgGs in vitro and in vivo. mAbs. 6:5, 1265-1273.
12. Jordan R E, et al. 2018 Proteinase-nicked IgGs: an unanticipated target for tumor immunotherapy. Oncoimmunology doi.org/10.1080
13. Kandarian et al, 2017, Flow Cytometry-Based Cytotoxicity Assay for the Assessment of Human NK Cell Activity. Journal of Visualized Experiments 126 e56191.
14. Bruggeman M G T et al. 1987 Comparison of the effector functions of human immunoglobulins using a matched set of chimeric antibodies. J. Exp. Med. 166, 1351-1361.
15. Ryan M. H. et al, 2007, Proteolysis of purified IgGs by human and bacterial enzymes in vitro and the detection of specific proteolytic fragments of endogenous IgG in rheumatoid synovial fluid. Mol. Immunol. 45, 1837-1846
16. Kinder et al. 2013 Engineered Protease-resistant Antibodies with Selectable Cell-killing Functions, J. Biol. Chem. 288, 30843-54.
17. Gene Cloning and DNA Analysis; Wiley-Blackwell 7th edition. Jan. 19, 2016: ISBN 10 9781119072560.
18. Brezski R J, Knight D M and Jordan R E. 2010. Cleavage of IgGs by proteases associated with invasive diseases; an evasion tactic against host immunity? mAbs 2:3; 1-9.
19. Hsiao H-C, Fan X, Jordan, RE, Zhang N, An Z. 2018. Proteolytic single hinge cleavage of pertuzumab impqirs its Fc effector function and antitumor activity in vitro and in vivo.
20. Zahavi D, Weiner L. 2020. Monoclonal Antibodies in Cancer Therapy. Antibodies 9, 34.
21. Zurawski D V, Mclendon M K. 2020. Monoclonal Antibodies as an Antibacterial Approach Against Bacterial Pathogens. Antibiotics, 9; 155.
22. Winsted et al. 2015 Jul. 15. Complete removal of extracellular IgG antibodies in a randomized dose-escalation Phase I study with the bacterial enzyme IdeS-a novel therapeutic opportunity. PLOS One. 10 (7): e0132011. doi: 10.1371/journal.pone.0132011. PMID: 26177518; PMCID: PMC4503742.
23. Sofia J. et al. 2017. Enzymatic Inactivation of Endogenous IgG by IdeS Enhances Therapeutic Antibody Efficacy. Molecular Cancer Therapeutics 16 (9), 1887-1897.
24. Jefferis, R. 2007. Antibody therapeutics: isotype and glycoform selection. Expert Opin. Biol. Ther. 7, 1401-1413.
25. Reff, M E et al. 1994. Depletion of B cells in Vivo by a Chimeric Mouse Human Monoclonal Antibody to CD20. Blood, 83; 435-445.
26. Punt, C J A et al. 1994. Anti-tumor antibody produced by human tumor-infiltrating and peripheral blood B lymphocytes. Cancer Immunology, Immunotherapy, 38; 225-232.
27. U.S. Pat. No. 8,133,483 B2. Mar. 13, 2012. Hansa Medical A B, Lund S E.

What is claimed is:

1. A method of treating a condition in a subject in need thereof, the method comprising administering to the subject a therapeutically effective amount of a modified IgG antibody, said modified IgG antibody comprising an epitope having at least 80% sequence similarity to a peptide having an amino acid sequence selected from the group consisting of the epitope sequences of SEQ ID NOs: 1-9.

2. The method of claim 1, wherein the condition treated is selected from the group consisting of: viral diseases, bacterial diseases, fungal diseases, parasitic diseases, and proliferative diseases other than cancer.

3. The method of claim 1, wherein the condition treated is cancer.

4. The method of claim 1, wherein the epitope comprises the sequence: PAPELLG (SEQ ID NO: 6) with a free C-terminus.

5. The method of claim 1, wherein the modified IgG antibody is generated by digesting an intact IgG antibody with an IdeS enzyme.

6. The method of claim 1, wherein the modified IgG antibody is genetically engineered to contain the epitope or wherein the modified IgG antibody is chemically modified to contain the epitope.

7. The method of claim 5, wherein the digestion is conducted for an amount of time and under conditions sufficient for IdeS proteolysis of the intact IgG to provide the IgG antibody comprising an IdeS-generated epitope.

8. The method of claim 5, wherein the digestion is conducted at 37° C. for about 10 minutes to about 24 hours wherein ratio of [intact IgG] to [IdeS] is between 1 to 0.001 w/w and 1 to 0.05 w/w, or about 1:0.02.

9. The method of claim 1, wherein the epitope is a neo-epitope to the subject.

10. The method of claim 5, wherein the modified IgG antibody is administered to the subject along with the IdeS enzyme.

11. The method of claim 5, wherein the IdeS enzyme is a recombinant IdeS.

12. The method of claim 5, wherein the intact IgG antibody is selected from the group consisting of: cetuximab, daratumumab, dinutuximab, elotuzumab, isatuximab, mogamulizumab, necitimumab, ofatumumab, olaratumumab, pertuzumab, ramucinumab, rituximab, trastuzumab and generic versions and or combinations thereof.

13. The method of claim 5, wherein the intact IgG antibody is selected from the group consisting of: anti-viral antibodies, anti-bacterial antibodies, anti-fungal antibodies, anti-parasitic antibodies.

14. The method of claim 1, wherein the subject in need thereof is a human.

15. The method of claim 1, wherein the subject comprises an endogenous anti-hinge antibody.

16. The method of claim 1, wherein the modified IgG antibody is a single-cleaved IgG (“scIgG”).

17. A method of treating cancer in a subject in need thereof, the method comprising co-administering a therapeutically effective amount of IdeS and a therapeutically effective amount of an IgG antibody in the subject, wherein the therapeutically effective amount of IdeS digests the IgG antibody or antibodies on the cancer cell surface to generate a modified IgG antibody comprising an IdeS-generated neo-epitope to which endogenous anti-hinge antibodies bind.

18. The method of claim 17, wherein the IgG antibody binds to a cancer specific antigen.

19. A method for treating cancer in a subject in need thereof, the method comprising administering a therapeutically effective amount of IdeS at a solid tumor site, wherein the IdeS digests endogenous anti-cancer autoantibodies at the solid tumor site to generate neo-epitope in the anti-cancer autoantibodies which attract endogenous anti-hinge antibodies, thereby enhancing antibody-dependent cellular cytotoxicity (ADCC) against the solid tumor.

20. The method of any of claim 19, wherein the subject in need thereof is a human.

21. The method of claim 1, wherein the activity of the IgG antibody comprising the Ides-generated epitope is 5 to 500 fold greater in vitro than an IgG antibody not treated with IdeS.
